(19) United States
(12) Patent Application Publication
(10) Pub. No.: US 2025/0114435 A1
(43) Pub. Date: Apr. 10, 2025

(54) ORAL DISPERSIBLE FILM OF SEMAGLUTIDE AND PREPARATION METHOD THEREOF

(71) Applicant: CorePharma, LLC

(72) Inventors: Arpit PATEL, Allentown, NJ (US); Rajendra NAGAMALLA, Kendall Park, NJ (US)

(21) Appl. No.: 18/908,838

(22) Filed: Oct. 8, 2024

Related U.S. Application Data
(60) Provisional application No. 63/589,039, filed on Oct. 10, 2023.

(51) Int. Cl. A61K 38/26; A61K 9/00; A61K 47/10; A61K 47/12; A61K 47/20; A61K 47/26; A61K 47/32; A61K 47/36; A61K 47/38; A61K 47/40

(52) U.S. Cl. CPC A61K 38/26; A61K 9/006; A61K 47/10; A61K 47/12; A61K 47/20; A61K 47/26; A61K 47/32; A61K 47/36; A61K 47/38; A61K 47/40

(57) ABSTRACT

The present invention is related to oral dispersible film of semaglutide for treating diabetes. The oral dispersible film of semaglutide comprising semaglutide, taste masking agent, permeation enhancer, film forming polymer, plasticizer, and other pharmaceutically acceptable excipients. The oral dispersible film of Semaglutide can be prepared by casting dispersion of semaglutide which is prepared in suitable solvent.

### TECHNICAL FIELD

The present disclosure relates to the field of pharmaceutical. The present disclosure in particular relates to an oral dispersible film of semaglutide with a pharmaceutical acceptable ingredients, a method of manufacturing the film and its use in the treatment of diabetes. The disclosed pharmaceutical composition comprising semaglutide that can be administered orally to a subject in need of treatment.

### BACKGROUND OF THE INVENTION

Diabetes mellitus (DM) is a disease of inadequate control of blood levels of glucose. There are two main sub classes, including type 1, and type 2. About 422 million people worldwide have diabetes, the majority living in low-and middle-income countries, and 1.5 million deaths are directly attributed to diabetes each year. Both the number of cases and the prevalence of diabetes have been steadily increasing over the past few decades.

Type 1 diabetes is an autoimmune disease. This means it begins when the body's immune system mistakenly attacks other cells in the body. In type 1 diabetes, the immune system destroys the insulin-producing cells (called beta cells) in the pancreas. This leaves the person with little or no insulin in his or her body.

Type 2 diabetes occurs when your body's cells become less responsive to insulin's efforts to drive glucose into the cells, a condition called insulin resistance. As a result, glucose starts to build up in the blood. Type 2 diabetes is much more common than type 1 diabetes. In the past 3 decades the prevalence of type 2 diabetes has risen dramatically in countries of all income levels.

Insulin is important for controlling the sugar concentration of the blood (blood glucose level) and is the only hormone in the body that can reduce the glucose level. In diabetes patients, chronically elevated blood sugar causes severe damage to the retina (diabetic retinopathy), sensory impairment of the nerves (neuropathy), and ketoacidosis (excess acidity due to deficient glucose utilization). However, an excessively low blood glucose level (hypoglycemia) can also cause unconsciousness (diabetic shock). For the patient's health, therefore, it is extremely important to maintain a balanced blood glucose level. Insulin can be administered for this purpose.

Glucagon-like peptide 1 (GLP-1) is a 30-amino acid peptide hormone produced in the intestinal epithelial endocrine L-cells. It has several roles, including triggering insulin release from your pancreas, blocking glucagon secretion, slowing stomach emptying, and increasing satiety.

GLP-1 agonists are a class of medications that mainly help manage blood sugar (glucose) levels in people with Type 2 diabetes. GLP-1 agonist medications work by mimicking this hormone. GLP-1 medications bind to GLP receptors to trigger the effects (or roles) of the GLP-1 hormone. The higher the dose of the GLP-1 agonist, the more extreme the effects. In Type 2 diabetes, the medications helps to manage your blood sugar by triggering your pancreas to release more insulin. The slowed digestion also helps decrease blood sugar spikes. The satiety effect of GLP1-agonists reduces your food intake, appetite and hunger. These combined effects often result in weight loss. GLP-1 agonist medications currently available on market are Dulaglutide, Exenatide, Exenatide, Liraglutide, Lixisenatide, and Semaglutide.

Semaglutide is a glucagon-like peptide 1 (GLP-1) analog used to manage type 2 diabetes along with lifestyle changes, such as dietary restrictions and increased physical activity. Semaglutide was developed by Novo Nordisk and approved by the FDA for subcutaneous injection in December 2017. The tablet formulation was approved for oral administration in September 2019. Semaglutide offers a competitive advantage over other drugs used to manage diabetes, which may require several daily doses. Oral administration of Semaglutide remains a challenge due to susceptibility to pH and gastric/small intestinal enzymes, as well as low intestinal epithelial membrane permeability. The low permeability results from minimal passive or carrier-mediated transcellular permeation across phospholipid bilayers, as well as restricted paracellular transport via tight junctions.

The insulin, as well as insulin analogs and other anti-diabetic peptides, are ordinarily administered to patients with diabetes mellitus by injection. However, this administration route is having some disadvantages like pain, swelling, redness, mild fever, etc. Hence, there is need to develop more dosage forms which can be administer by oral route. However, the orally-administered insulin is rapidly degraded by proteolytic enzymes the digestive tract. Hence, there are challenges in developing orally administrable anti-diabetic drugs. Despite the obstacles occurred by problems connected with oral administration of insulin, efforts have been made to develop oral insulin and oral anti-diabetic dosage forms.

Currently, in the market semaglutide conventional tablet, extended release tablet and injectable are available. Hence, there is need to develop such formulation which can provide the most convenient mode of administration, rapidly dissolving, giving quicker onset of action, and enhanced permeability and bioavailability compared to other oral dosage forms.

### BRIEF SUMMARY OF THE INVENTION

Disclosed is an oral dispersible film and process preparation thereof. The oral dispersible film of the present invention is fast disintegrating and suitable for administration to all age groups.

The main objective and aspect of the present invention is to provide an oral dispersible film of semaglutide.

The another objective of the present invention is to mask the unpleasant taste of semaglutide with different techniques like by using regular sweeteners, flavours and bitter blockers or by using complexation methods or by using polymeric coating methods.

The one more objective of the present invention is to provide an oral dispersible film of semaglutide which increases permeability of the drug.

As per one aspect of the present invention an oral dispersible film of semaglutide comprising semaglutide, taste masking agent, permeation enhancer, film forming polymer, plasticizer, and other pharmaceutically acceptable excipients.

As per one more aspect, the present invention provides process of preparation of an oral dispersible film of semaglutide comprising dispersing semaglutide in suitable solvent followed by addition of taste masking agent and other excipients; mixing to get uniform dispersion; casting dispersion on base substrate; drying with hot air; cutting in specific size thereby forming oral dispersible film of semaglutide.

As per another aspect, the present invention provides a method for treating diabetes in a subject in need of such treatment by administering an oral dispersible film of Semaglutide.

The proposed oral film dosage has excellent effects of masking a bitter taste and may be easily prepared by a simple process at low cost, which achieves ideal dissolution, uniformity, stability, and an orally disintegrating hence enabling rapid absorption of drug and increases permeability and bioavailability.

### DETAILED DESCRIPTION OF THE INVENTION

It is understood that the present invention is not limited to the particular methodology, protocols, reagents, etc. described herein, as these may vary. It is also to be understood that the terminology used herein is used for the purpose of describing particular embodiments only, and is not intended to limit the scope of the present invention. It must be noted that as used herein and in the appended embodiments, the singular forms “a,” “an,” and “the” include plural reference unless the context clearly dictates otherwise.

Unless defined otherwise, all technical and scientific terms used herein have the same meanings as commonly understood by one of ordinary skill in the art to which this invention belongs. Preferred methods, system components, and materials are described, although any methods and materials similar or equivalent to those described herein can be used in the practice or testing of the present invention. All references cited herein are incorporated by reference herein in their entirety.

As stated herein, that it follows in a transitional phrase or in the body of a claim, the terms “comprise(s)” and “comprising” are to be interpreted as having an open ended meaning. When used in the context of a process, the term “comprising” means that the process includes at least the recited steps, but may include additional steps. When used in the context of a composition, the term “comprising” means that the composition includes at least the recited features or components, but may also include additional features or components.

As used herein, the term “about”, when referring to a value or to an amount of mass, weight, time, linear-dimension, volume, concentration, and/or percentage can encompass variations of, in some embodiments +/−20%, in some embodiments +/−10%, in some embodiments +/−5%, in some embodiments +/−1%, in some embodiments +/−0.5%, and in some embodiments +/−0.1%, from the specified amount, as such variations are appropriate in the disclosed packages and methods.

The term “oral dispersible film” refers to the drug delivery systems that they are quickly releasing the drug by dissolving or dispersing in the mucosa with saliva within a few seconds when it placed in the mouth cavity or on the tongue”.

The term “stable” refers to a product which exhibits no changes in the dissolution profile or remains within the established specifications and recovery when the product is exposed to normal stability conditions (e.g., 25° C./60% RH and 40° C./75% RH) for an extended period of time.

The term “bitter blockers” bitter-blockers refers to compounds which modify bitter taste by interacting with the bitter-taste perception pathway in some way, acting at a pharmacological level; interfering with taste receptors or the taste-transduction mechanism. The terms “bitter blockers” and “taste masking agent” can be interchangeably used.

The term “processing solvent” used herein refers to a substance with the ability to dissolve other substances to form a solution. The terms “processing solvent” and “solvent” can be interchangeably used.

The term “permeation enhancer” used herein refers to chemical compounds that can facilitate the penetration of active pharmaceutical ingredients into or through poorly permeable biological membranes. The terms “permeation enhancer” and “penetration enhancer” can be interchangeably used.

The present invention relates to the oral dispersible film and method of manufacture of orally-dispersible, edible, or consumable films as a vehicle for the non-invasive administration of semaglutide through the mucosal tissues of the oral cavity including, but not limited to, the mouth, pharynx, and oesophagus.

In the main embodiment of the present invention, an oral dispersible film of semaglutide comprises semaglutide, a taste masking agent, a permeation enhancer, a film forming polymer, a plasticizer, other pharmaceutically acceptable excipients, and a processing solvent.

As per one embodiment of the present invention, semaglutide is present in an amount from about 1% w/w to about 50% w/w, preferably from about 1% w/w to about 40% w/w, more preferably from about 1% w/w to about 30% w/w, more preferably from about 1% w/w to about 25% w/w most preferably 1% w/w to about 20% w/w.

As per one embodiment of the present invention, permeation enhancer include, but not limited to, N-acetyl Cysteine, Propylene Glycol, TPGS (d-α-Tocopheryl polyethylene glycol 1000 succinate), Tween 20, medium-chain fatty acids like caprylic acid or caprate, or its amino acid ester like Salcaprozate sodium, menthol, an ionic surfactant, a nonionic surfactant, a polysorbate, a derivative of tocopherol, a poloxamer, a monoglyceride, a diglyceride, a fatty acid, or a fatty alcohol like MCT or a combination thereof.

As per the preferred embodiment of the present invention, Medium Chain Triglycerides (MCT) is used as a permeation enhancer.

As per one embodiment of the present invention, the permeation enhancer is in an amount from about 0.01% w/w to about 30% w/w, preferably from about 0.01% w/w to about 20% w/w, more preferably 0.01% w/w to about 10% w/w, more preferably from about 0.05% w/w to about 10% w/w, most preferably from 0.1% w/w to about 10% w/w.

As per one embodiment of the present invention, film forming polymer can be selected from hydrophilic or hydrophobic polymer.

As per one embodiment of the present invention, hydrophobic polymers can be selected from include, but not limited to, acrylics, epoxies, polyethylene, polystyrene, polyvinylchloride, polytetrafluorethylene, polydimethylsiloxane, polyesters, and polyurethanes, chitosan.

As per one embodiment of the present invention, hydrophilic polymer can be selected from include, but not limited to, polyvinyl alcohol (PVA), hydroxypropyl cellulose (HPC), hydroxyethyl cellulose, hydroxypropyl methyl cellulose, hypromellose (HPMC), sodium carboxymethyl cellulose (CMC-Na), xanthan gum, pectin, copovidone, povidone, guar gum, pullulan, polyethylene oxide, and sodium alginate.

As per the preferred embodiment of the present invention, hydrophilic polymer, Hydroxy propyl methyl cellulose is used as a film forming polymer.

As per one embodiment of the present invention, film forming polymer is in an amount from about 10% w/w to about 60% w/w, preferably from about 10% w/w/to about 50% w/w, more preferably from about 10% w/w to about 55% w/w, more preferably from about 10% w/w to about 40% w/w preferably most preferably 10% w/w to about 30% w/w.

As per one embodiment of the present invention, plasticizers can be selected from sorbitol, maltitol, xylitol, glycerin, polyethylene glycol, propylene glycol, hydrogenated starch syrup, starch syrup, triacetin, glycerol oleate, sucrose fatty acid ester and double chain fatty acid.

As per the preferred embodiment of the present invention, propylene glycol is used as a plasticizer.

As per one embodiment of the present invention, a plasticizer is in an amount from about 0.1% w/w to about 20% w/w, preferably from about 0.1% to about 15% w/w, more preferably from about 0.1% to about 14% w/w, more preferably from about 0.1% to about 12% w/w, most preferably from about 0.1% to about 10% w/w.

As per one embodiment of the present invention, taste masking agent can be selected from the group comprising sweeteners, flavours, bitter blockers, complexation agent and coating polymers.

As per one embodiment of the present invention, bitter blockers used as a taste-masking agent can be selected from adenosine monophosphate, lipoproteins, phospholipids, haumatin, aspartame, monellin and neotame.

As per one embodiment of the present invention, polymers used as a taste-masking agent can be selected from hydrophilic or hydrophobic polymers, but not limited to Eudragit E-100, ethyl cellulose, HPMC, HPC, polyvinyl alcohol, and polyvinyl acetate.

As per one embodiment of the present invention, complexation agent used as taste-masking agent includes but not limited to cyclodextrin and ion exchange resins.

As per the preferred embodiment of the present invention, beta cyclodextrin is used as taste masking agent.

As per one embodiment of the present invention, taste masking agent is in amount from about 0.1% w/w to about 30% w/w, preferably from about 0.5% to about 30% w/w, more preferably from about 0.5% to about 25% w/w, more preferably from about 0.8% to about 25% w/w, most preferably from about 1% to about 25% w/w.

As per one embodiment of the present invention, the other pharmaceutically acceptable excipients can be selected from but not limited to sweetening agent, flavouring agent, saliva stimulating agent, colouring agent, antioxidant, diluent.

As per one embodiment of the present invention, sweetening agent can be selected from sucrose, acesulfame potassium, glucose, maltose, oligosaccharides dextrin, alpha cyclodextrin, beta cyclodextrin, gamma cyclodextrin, hydroxypropyl beta cyclodextrin, cluster dextrin, indigestible dextrin, hydrogenated indigestible dextrin, invert sugar, fructose, lactose, galactose, starch syrup, sorbitol, maltitol, xylitol, erythritol, hydrogenated starch syrup, mannitol, trehalose, and combinations thereof.

As per the preferred embodiment of the present invention, combination of sucrose and acesulfame potassium is used as taste masking agent.

As per one embodiment of the present invention, sweetening agent is in amount from about 0.1% w/w to about 20% w/w, preferably from about 0.1% to about 15% w/w, more preferably from about 0.1% to about 14% w/w, more preferably from about 0.1% to about 12% w/w, most preferably from about 0.1% to about 10% w/w.

As per one embodiment of the present invention, flavouring agent may natural flavour, artificial flavour or a mixture thereof. The natural flavour may include aromatic plants, especially, extract and/or oil obtained from leaves, flowers, and fruits of the aromatic plants. The plant oil may include spearmint oil, cinnamon oil, peppermint oil, lemon oil, clove oil, bay oil, thyme oil, cedar leaf oil, nutmeg oil, sage oil, almond oil, and the like. The artificial flavour may include synthetic fruit flavours such as lemon, orange, grape, lime, strawberry, etc. and other synthetic flavours such as vanilla, peppermint, watermelon, chocolate, coffee, cocoa, pine leaf, ginseng, red ginseng, citrus, etc.

As per the preferred embodiment of the present invention, peppermint is used as flavouring agent.

As per one embodiment of the present invention, flavouring agent is in amount from about 0.1% w/w to about 20% w/w, preferably from about 0.1% to about 18% w/w, more preferably from about 0.1% to about 15% w/w, more preferably from about 0.1% to about 12% w/w, most preferably from about 0.1% to about 10% w/w.

As per one embodiment of the present invention, saliva stimulating agents include food acids Such as citric, lactic, malic, succinic, ascorbic, adipic, fumaric and tartaric acids.

As per the preferred embodiment of the present invention, citric acid is used as saliva stimulating agent.

As per one embodiment of the present invention, saliva stimulating agent is in amount from about 0.1% w/w to about 20% w/w, preferably from about 0.1% to about 18% w/w, more preferably from about 0.1% to about 15% w/w, more preferably from about 0.1% to about 10% w/w, most preferably from about 0.1% to about 5% w/w.

As per one embodiment of the present invention, coloring agents are added to the film-forming materials to improve the colour of the oral soluble film. The coloring agent can be selected from but not limited to the group consisting of FD&C colours, D&C colours like blue, orange, yellow, purple, green and combinations thereof.

As per the preferred embodiment of the present invention, blue colour is used as coloring agent.

As per one embodiment of the present invention, coloring agent is in amount from about 0.001% w/w to about 2% w/w, preferably from about 0.001% to about 1% w/w, more preferably from about 0.005% to about 1% w/w, more preferably from about 0.008% to about 1% w/w, most preferably from about 0.008% to about 0.5% w/w.

As per one embodiment of the present invention, solubilizer can be selected from povidone, Copovidone, polyethylene glycol, polysorbates, stearic acid, soya lecithin, sorbitan monostearate, and polyvinylalcohol.

As per the preferred embodiment of the present invention, Copovidone (Kollidon VA 64) is used as solubilizer.

As per one embodiment of the present invention, solubilizer is in amount from about 1% w/w to about 30% w/w, preferably from about 1% to about 25% w/w, more preferably from about 5% to about 25% w/w, more preferably from about 10% to about 25% w/w, most preferably from about 15% to about 25% w/w.

As per one embodiment of the present invention, antioxidants can be selected from ascorbic acid, Butylated hydroxyanisole, Butylated hydroxytoluene, Ascorbyl palmitate, Potassium metabisulfite, Propyl gallate, Sodium metabisulfite, Sodium sulphite, tartaric acid, and tocopherol.

As per the preferred embodiment of the present invention, Butylated hydroxyanisole or Butylated hydroxytoluene is used as antioxidant.

As per one embodiment of the present invention, antioxidant is in amount from about 0.01% w/w to about 5% w/w, preferably from about 0.001% to about 1% w/w, more preferably from about 0.005% to about 1% w/w, more preferably from about 0.008% to about 1% w/w, most preferably from about 0.05% to about 0.5% w/w.

As per one embodiment of the present invention, diluents can be selected from starch, Starch 1500, lactose, mannitol, dextrose, mannitol, sodium starch glycolate, pregelatinized starch.

As per the preferred embodiment of the present invention, Pregelatinized starch (starch 1500) is used as diluent.

As per one embodiment of the present invention, diluent is in amount from about 1% w/w to about 20% w/w, preferably from about 1% to about 18% w/w, more preferably from about 1% to about 15% w/w, more preferably from about 1% to about 12% w/w, most preferably from about 1% to about 10% w/w.

As per one embodiment of the present invention, solvent can be selected from but not limited to organic solvents such as alcohols, ketones, etc, or water or mixtures thereof can be used.

As per the preferred embodiment of the present invention, water and acetone are used as solvent.

As per one embodiment of the present invention, the oral dispersible film of semaglutide comprises from about 1 to about 50% w/w semaglutide; from about 0.1 to about 30% w/w taste masking agent; from about 0.01 to about 30% w/w permeation enhancer; from 10% w/w to about 60% w/w film forming polymer; from 0.1% w/w to about 20% w/w plasticizer; from 1% w/w to about 20% w/w diluent; from 1% w/w to about 30% w/w solubilizer; from 0.1% w/w to about 20% w/w sweetening agent; from 0.1% w/w to about 5% w/w antioxidant; from 0.1% w/w to about 20% w/w saliva stimulating agent; from 0.001% w/w to about 2% w/w colouring agent; from 0.1% w/w to about 20% w/w flavouring agent; and a processing solvent.

As per one embodiment of the present invention, the oral dispersible film of semaglutide comprises from about 1% w/w to about 20% w/w semaglutide; from about 1% w/w to about 25% w/w taste masking agent; from about 0.1 to about 10% w/w permeation enhancer; from 10% w/w to about 30% w/w film forming polymer; from 0.1% w/w to about 10% w/w plasticizer; from 1% w/w to about 10% w/w diluent; from 15% w/w to about 25% w/w solubilizer; from 0.1% w/w to about 10% w/w sweetening agent; from 0.05% w/w to about 1% w/w antioxidant; from 0.1% w/w to about 5% w/w saliva stimulating agent; from 0.008% w/w to about 0.5% w/w colouring agent; from 0.1% w/w to about 10% w/w flavouring agent; and a processing solvent.

As per one embodiment of the present invention, the process of preparation of an oral dispersible film of semaglutide comprises the steps of:
a) Heating purified water and dissolving solubilizer and taste masking agent in heated water to get clear solution;
b) Keeping aside solution of step (a) to cool down at room temperature;
c) Adding and mixing Semaglutide and other excipients in step (b) solution to get uniform dispersion;
d) Subjecting dispersion of step (c) for homogenization and de-aeration;
e) Transferring the dispersion of step (d) to coating trough;
f) Coating the dispersion of step e) with film forming polymer with defined thickness on PET liner using lab coater instrument;
g) Drying coated dispersion of step (f) in hot air oven;
h) Cutting the dried dispersion of step (g) in specific size thereby forming an oral dispersible film of Semaglutide.

As per one embodiment of the present invention, oral dispersible film of semaglutide useful in the treatment of diabetes mellitus type 2.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention. The following examples, which include preferred embodiments, will serve to illustrate the practice of this invention, it being understood that the particulars shown are by way of example and for illustrative discussion of preferred embodiments of the invention.

### EXAMPLES

### Example 1: FORMULATION OF AN ORAL DISPERSIBLE FILM OF SEMAGLUTIDE (B1)

TABLE 1
Formulation of an Oral Dispersible Film of Semaglutide (B1)

| Sr. No. | Name of the ingredients | % w/w |
|---|---|---|
| 1 | Semaglutide | 1-50% |
| 2 | Taste Masking Agent | 0.1-30% |
| 3 | Permeation Enhancer | 0.01-30% |
| 4 | Film Forming Agent | 10-60% |
| 5 | Diluent | 1-20% |
| 6 | Solubilizer | 1-30% |
| 7 | Sweetening agent | 0.1-20% |
| 8 | Plasticizer | 0.1-20% |
| 9 | Antioxidant | 0.1-5% |
| 10 | Saliva stimulating Agent | 0.1-20% |
| 11 | Colouring Agent | 0.01-1% |
| 12 | Flavouring Agent | 0.1-20% |
| 13 | Processing Solvent | Q.S. |
| | Total weight | 100.00 |

### Procedure:

As per procedure mentioned under example 4

### Example 2: FORMULATION OF AN ORAL DISPERSIBLE FILM OF SEMAGLUTIDE (B2)

TABLE 2
Formulation of an Oral Dispersible Film of Semaglutide (B2)

| Sr. No. | Name of the ingredients | % w/w |
|---|---|---|
| 1 | Semaglutide | 1-20% |
| 2 | Taste Masking Agent | 1-25% |
| 3 | Permeation Enhancer | 0.1-10% |
| 4 | Film Forming Agent | 10-30% |
| 5 | Diluent | 1-10% |
| 6 | Solubilizer | 15-25% |
| 7 | Sweetening agent | 0.1-10% |
| 8 | Plasticizer | 0.1-10% |
| 9 | Antioxidant | 0.05-1% |
| 10 | Saliva stimulating Agent | 0.1-5% |
| 11 | Colouring Agent | 0.008-0.5% |
| 12 | Flavouring Agent | 0.1-10% |
| 13 | Processing Solvent | Q.S. |
| | Total weight | 100.00 |

### Procedure:

As per procedure mentioned under example 4

### Example 3: FORMULATION OF AN ORAL DISPERSIBLE FILM OF SEMAGLUTIDE (B3)

TABLE 3
Formulation of an Oral Dispersible Film of Semaglutide (B3)

| Sr. | | | Qty./film | % |
|---|---|---|---|---|
| No | Name of the Ingredients | Role | (in mg) | w/w |
| 1 | Semaglutide | API | 14.00 | 14.00 |
| 2 | Beta Cyclodextrin | Taste Masking Agent | 20.00 | 20.00 |
| 3 | MCT | Permeation Enhancer | 5.00 | 5.00 |
| 4 | Hydroxy propyl methyl | Film Forming Agent | 24.00 | 24.00 |
| | cellulose | | | |
| 5 | Pregelatinized starch or | Diluent | 4.89 | 4.89 |
| | Sodium Starch Glycolate | | | |
| 6 | Copovidone | Solubilizer | 20.00 | 20.00 |
| 7 | Sucralose | Sweetening agent | 2.00 | 2.00 |
| 8 | Acesulfame potassium | Sweetening agent | 3.00 | 3.00 |
| 9 | Propylene glycol | Plasticizer | 3.00 | 3.00 |
| 10 | Butylated hydroxytoluene | Antioxidant | 0.10 | 0.10 |
| | (BHT) or Butylated | | | |
| | hydroxyanisole (BHA) | | | |
| 11 | Citric acid | Saliva stimulating | 1.00 | 1.00 |
| | | Agent | | |
| 12 | Colouring Agent | Colouring Agent | 0.01 | 0.01 |
| 13 | Flavouring Agent | Flavouring Agent | 3.00 | 3.00 |
| 14 | Solvent | Processing solvent | — | — |
| | Total weight | | 100.00 | 100.00 |

### Procedure:

As per procedure mentioned under example 4

### Example 4: FORMULATION OF AN ORAL DISPERSIBLE FILM OF SEMAGLUTIDE (B4)

TABLE 4
Formulation of an Oral Dispersible Film of Semaglutide (B4)

Quantity per film

| Batch B4 | mg | % w/w |
|---|---|---|
| Semaglutide | 3.00 | 7.89 |
| Beta cyclodextrin | 3.00 | 7.89 |
| Copovidone | 1.00 | 2.63 |
| Hydroxy propyl methyl cellulose | 19.96 | 52.53 |
| Sodium starch glycolate | 2.00 | 5.26 |
| Sucralose | 1.00 | 2.63 |
| Acesulfame pottasium | 1.00 | 2.63 |
| Propylene glycol | 6.00 | 15.79 |
| Butylated hydroxytoluene (BHT) | 0.03 | 0.08 |
| Blue Colour | 0.01 | 0.03 |
| Watermelon Flavour | 1.00 | 2.63 |
| Purified water | Q.S. | |
| Acetone | Q.S. | — |
| Total Weight | 310.00 | 100.00 |

### Procedure:

a) Heating purified water up to 60° C. and dissolving Kollidon VA 64 and Beta cyclodextrin in heated water to get clear solution;
b) Keeping aside solution of step (a) to cool down at room temperature;
c) Adding and mixing Semaglutide in step (b) solution to get clear solution;
d) Adding and mixing Acesulfame pottasium and sucralose in step (c) solution;
e) Adding and mixing other excipients except Butylated hydroxytoluene in step (d) solution;
f) Mixing Butylated hydroxytoluene in acetone in a separate vessel to get clear solution;
g) Mixing of step (d) and step (e) solution to get uniform dispersion;
h) Subjecting dispersion of step (g) for homogenization and de-aeration;
i) Transferring the dispersion of step (h) to coating trough;
j) Coating the dispersion of step (i) with Hydroxy propyl methyl cellulose polymer with defined thickness on PET liner using lab coater instrument;
k) Drying coated dispersion of step (j) in hot air oven;
l) Cutting the dried dispersion of step (k) in specific size thereby forming an oral dispersible film of Semaglutide.

### Coating & Drying Parameters:

| | |
|---|---|
| Parameters | Set values |

Trial coat

| Coating thickness | 550μ | |
|---|---|---|
| Drying temperature | 40° | C. |
| Drying time | 25 | min. |
| Average Weight | 31.2 | mg |

Final coat

| Coating thickness | 650μ | |
|---|---|---|
| Drying temperature | 40° | C. |
| Drying time | 29 | min. |
| Average Weight | 39.2 | mg |

### Example 5: EVALUATION OF AN ORAL DISPERSIBLE FILM OF SEMAGLUTIDE

The oral dispersible film of semaglutide was evaluated for physical appearance, strip surface pH, assay, disintegration time, and dissolution profile.

### Result:

| | | Result |
|---|---|---|
| Sr. No. | Test Parameter | B4 |
| 1 | Appearance | Red coloured |
| | | rectangular shape |
| 2 | strip surface pH | 7.0 |
| 3 | Assay | 93.13 |
| 4 | Disintegration time (in Sec) | 20 |
| 5 | Dissolution (in %) | 134 |

From the study data it can be concluded that the oral films of the present invention satisfies all the parameters of good film.

We claim:

1. An oral dispersible film of semaglutide comprises semaglutide, a taste masking agent, a permeation enhancer, a film forming polymer, a plasticizer, other pharmaceutically acceptable excipients, and a processing solvent.

2. The oral dispersible film of semaglutide as claimed in claim 1 wherein said semaglutide can be present in the formulation of the present invention in an amount from about 1% w/w to about 50% w/w, more preferably from about 1% w/w to about 30% w/w, and most preferably in the range from about 1% w/w to about 20% w/w.

3. The oral dispersible film of semaglutide as claimed in claim 1 wherein said taste masking agent can be selected from the group comprising sweeteners, flavours, bitter blockers complexation agent and coating polymers.

4. The oral dispersible film of semaglutide as claimed in claim 1 wherein said taste masking agent present in an amount from 0.1% w/w to about 30% w/w, most preferably from 1% w/w to about 25% w/w.

5. The oral dispersible film of semaglutide as claimed in claim 1 wherein said permeation enhancer can be selected from N-acetyl Cysteine, Propylene Glycol, TPGS (d-α-Tocopheryl polyethylene glycol 1000 succinate), Tween 20, medium-chain fatty acids like caprylic acid or caprate, or its amino acid ester like Salcaprozate sodium, menthol, an ionic surfactant, a nonionic surfactant, a polysorbate, a derivative of tocopherol, a poloxamer, a monoglyceride, a diglyceride, a fatty acid or fatty alcohol like MCT or a combination thereof.

6. The oral dispersible film of semaglutide as claimed in claim 1 wherein said permeation enhancer is present in an amount from 0.01% w/w to about 30% w/w, most preferably from 0.1% w/w to about 10% w/w.

7. The oral dispersible film of semaglutide as claimed in claim 1 wherein said film forming polymer can be selected from hydrophilic or hydrophobic polymer,
wherein said hydrophobic polymer can be selected from include, but not limited to, acrylics, epoxies, polyethylene, polystyrene, polyvinyl chloride, polytetrafluorethylene, polydimethylsiloxane, polyesters, and polyurethanes, chitosan and
wherein said hydrophilic polymer can be selected from include, but not limited to, polyvinyl alcohol (PVA), hydroxypropyl cellulose (HPC), hydroxyethyl cellulose, hydroxypropyl methylcellulose, hypromellose (HPMC), sodium carboxymethyl cellulose (CMC-Na), xanthan gum, pectin, copovidone, povidone, guar gum, pullulan, polyethylene oxide, and sodium alginate.

8. The oral dispersible film of semaglutide as claimed in claim 1 wherein said film forming polymer is present in an amount from 10% w/w to about 60% w/w, most preferably from 10% w/w to about 30% w/w.

9. The oral dispersible film of semaglutide as claimed in claim 1 wherein said plasticizer can be selected from sorbitol, maltitol, xylitol, glycerin, polyethylene glycol, propylene glycol, hydrogenated starch syrup, starch syrup, triacetin, glycerol oleate, sucrose fatty acid ester and double chain fatty acid.

10. The oral dispersible film of semaglutide as claimed in claim 1 wherein said plasticizer is present in an amount from 0.1% w/w to about 20% w/w, most preferably from 0.1% w/w to about 10% w/w.

11. The oral dispersible film of semaglutide as claimed in claim 1 wherein said other pharmaceutically acceptable excipients can be selected from the group comprising sweetening agent, flavouring agent, saliva stimulating agent, colouring agent, solubilizer, antioxidant, diluent or combinations thereof.

12. The oral dispersible film of semaglutide as claimed in claim 1 wherein said processing solvent can be selected from organic solvents such as alcohols, ketones, etc, or water or mixtures thereof.

13. The oral dispersible film of semaglutide as claimed in claim 1, can be useful in the treatment of diabetes mellitus type 2.

14. The oral dispersible film of semaglutide as claimed in claim 1 comprises from about 1 to about 50% w/w semaglutide; from about 0.1 to about 30% w/w taste masking agent; from about 0.01 to about 30% w/w permeation enhancer; from 10% w/w to about 60% w/w film forming polymer; from 0.1% w/w to about 20% w/w plasticizer; from 1% w/w to about 20% w/w diluent; from 1% w/w to about 30% w/w solubilizer; from 0.1% w/w to about 20% w/w sweetening agent; from 0.1% w/w to about 5% w/w antioxidant; from 0.1% w/w to about 20% w/w saliva stimulating agent; from 0.001% w/w to about 2% w/w colouring agent; from 0.1% w/w to about 20% w/w flavouring agent; and a processing solvent.
